(19) United States
(12) Patent Application Publication
(10) Pub. No.: US 2023/0380796 A1
(43) Pub. Date: Nov. 30, 2023

(54) ULTRASONIC IMAGING SYSTEM AND VISCOSITY QUALITY CONTROL METHOD

(71) Applicant: SHENZHEN MINDRAY BIO-MEDICAL ELECTRONICS CO., LTD., Shenzhen (CN)

(72) Inventors: Xiao CHEN, SHENZHEN (CN); Shuangshuang LI, SHENZHEN (CN)

(21) Appl. No.: 18/202,154

(22) Filed: May 25, 2023

(30) Foreign Application Priority Data
May 27, 2022 (CN) 202210593357.X

(51) Int. Cl. A61B 8/08

(52) U.S. Cl. CPC A61B 8/08; A61B 8/485; A61B 8/469

(57) ABSTRACT

Disclosed are an ultrasonic imaging system and a viscosity quality control method, in which ultrasonic waves for detecting shear waves propagated in a region of interest are transmitted to the region of interest to obtain ultrasonic echo signals, a frequency dispersion distribution diagram is calculated based on the ultrasonic echo signals, a viscosity parameter is calculated based on the frequency dispersion distribution diagram, and the viscosity parameter is performed with quality control based on the frequency dispersion distribution diagram. The present disclosure provides a scheme of quality control on the viscosity parameter.

### CROSS-REFERENCE TO RELATED APPLICATIONS

This application is based on and claims priority to and benefits of Chinese Patent Application No. 202010593357.X, filed on May 27, 2022. The entire content of the above-referenced application is incorporated herein by reference.

### TECHNICAL FIELD

The present disclosure relates to ultrasonic imaging systems and viscosity quality control methods.

### BACKGROUND

Ultrasonic elasticity imaging technology, a research hotspot in the field of ultrasonic imaging in the past two decades, is related to non-invasive auxiliary diagnosis of major diseases such as breast cancer and liver cirrhosis by extracting tissue hardness related information. After years of development, ultrasonic elastic imaging techniques have been gradually mature, and has been widely used in clinical research and auxiliary diagnosis of various parts of human body (such as liver, breast, thyroid, musculoskeletal, vascular, prostate, cervix, etc.) in recent years. It can reflect qualitatively the soft and hard difference of the lesion relative to the surrounding tissue, or reflect quantitatively the physical parameters related to the hardness of the target tissue, such as Young's modulus, shear modulus, etc., which is welcomed widely by doctors.

Common ultrasonic elasticity imaging techniques include strain elasticity imaging, transient elasticity imaging and shear-wave elasticity imaging which is especially the latest one. Shear wave elasticity imaging is performed by transmitting special pulses into tissues to produce acoustic radiation force to generate the propagation of shear wave, then detecting and recording the propagation process of shear wave by ultrasonic wave, further calculating the propagation velocity of shear wave, and finally obtaining elastic modulus parameters reflecting the hardness of the tissues to achieve quantitative elasticity imaging. This technique has greatly expanded the clinical application field of elastic imaging, attracting great research interest.

In most current studies about elasticity imaging, tissues are regarded as a pure elastic body, and based on the assumption of which, elasticity imaging is performed to obtain a corresponding imaging result, i.e. the elasticity result. Especially for the quantitative elasticity imaging technique, only elastic modulus is calculated for display. However, a growing number of studies have shown that in addition to elasticity, human tissue also has the property of viscosity. Elasticity and viscosity jointly affect the propagation velocity of shear waves in tissues. Therefore, if the information about the viscosity of tissues can be extracted, it will have great clinical potential value; however, before the extracted information about the viscosity of tissues is clinically applied, there is a need to solve a problem that the extracted information about the viscosity of tissues is reliable.

### SUMMARY

In view of the above problems, the present disclosure provides an ultrasonic imaging system and a viscosity quality control method for quality control of a calculated viscosity parameter, which is described in detail below.

According to a first aspect, a viscosity quality control method provided in an embodiment may include:
transmitting ultrasonic waves for detecting shear waves to obtain ultrasonic echo signals, the shear waves being propagated in a region of interest;
calculating a frequency dispersion distribution diagram according to the ultrasonic echo signals;
calculating a viscosity parameter according to the frequency dispersion distribution diagram;
obtaining a viscosity quality control characteristic of the viscosity parameter according to the frequency dispersion distribution diagram; and
displaying viscosity quality control information about the viscosity parameter according to the viscosity quality control characteristic.

In an embodiment, the viscosity quality control characteristic comprises any one or more of the following characteristic quantities:
an effective frequency range for calculating the viscosity parameter;
a degree of matching when performing model fitting for the viscosity parameter according to the frequency dispersion distribution diagram;
a continuity of a frequency dispersion curve in the frequency dispersion distribution diagram;
a signal-to-noise ratio of the frequency dispersion distribution diagram; and
different shear waves in multiple patterns in the frequency dispersion distribution diagram.

In an embodiment, displaying viscosity quality control information about the viscosity parameter according to the viscosity quality control characteristic comprises:
plotting the viscosity quality control characteristic on the frequency dispersion distribution diagram to generate a frequency dispersion characteristic graph; and
displaying the frequency dispersion characteristic graph.

In an embodiment, plotting the viscosity quality control characteristic on the frequency dispersion distribution diagram to generate a frequency dispersion characteristic graph comprises:
marking the effective frequency range on the frequency dispersion distribution diagram when the viscosity quality control characteristic comprises the effective frequency range, or marking the effective frequency range and a target frequency range used for calculating the viscosity parameter on the frequency dispersion distribution diagram;
and/or
obtaining a fitted line in calculation of the viscosity parameter when the viscosity quality control characteristic comprises the degree of matching, and plotting the fitted line on the frequency dispersion distribution diagram;
and/or
connecting only continuous points of the frequency dispersion curve in the frequency dispersion distribution diagram so as to plot the frequency dispersion curve when the viscosity quality control characteristic comprises the continuity of the frequency dispersion curve in the frequency dispersion distribution diagram;
and/or
extracting dispersion curves for respective shear waves in multiple patterns and plotting the extracted dispersion curves on the frequency dispersion distribution diagram when the viscosity quality control characteristic comprises the different shear waves in multiple patterns in the frequency dispersion distribution diagram.

In an embodiment, before plotting the viscosity quality control characteristic on the frequency dispersion distribution diagram, foreground feature enhancement or background fading process may be performed on the frequency dispersion distribution diagram.

In an embodiment, displaying the viscosity quality control information about the viscosity parameter according to the viscosity quality control characteristic comprises:
displaying the viscosity quality control information about the viscosity parameter by a value characterizing the viscosity quality control characteristic.

In an embodiment, displaying the viscosity quality control information about the viscosity parameter by a value characterizing the viscosity quality control characteristic comprises:
when the viscosity quality control characteristic comprises the effective frequency range, displaying a value of the effective frequency range, or displaying a value of the effective frequency range and a value of a target frequency range used for calculating the viscosity parameter, or calculating and displaying an overlapping degree of the effective frequency range and a target frequency range;
and/or
calculating a fitted line of the viscosity parameter and a degree of fit between data used for fitting when the viscosity quality control characteristic comprises the degree of matching, and displaying the degree of fit comprising an average absolute difference value, a mean square error, a root mean square error, a coefficient of determination R2 or a correlation coefficient;
and/or
calculating and displaying a proportion of continuous or discontinuous segments in the frequency dispersion curve when the viscosity quality control characteristic comprises the continuity of the frequency dispersion curve in the frequency dispersion distribution diagram;
and/or
calculating and displaying a signal-to-noise ratio of the frequency dispersion distribution diagram when the viscosity quality control characteristic comprises the signal-to-noise ratio of the frequency dispersion distribution diagram;
and/or
when the viscosity quality control characteristic comprises the different shear waves in multiple patterns in the frequency dispersion distribution diagram, calculating and displaying a number of the different shear waves in multiple patterns in the frequency dispersion distribution diagram, or, determining a main shear wave and calculating and displaying a degree of influence of other pattern waves on the main shear wave, wherein the degree of influence of other pattern waves on the main shear wave comprises a proportion of energy of other pattern waves or main shear wave.

In an embodiment, displaying the viscosity quality control information about the viscosity parameter according to the viscosity quality control characteristic comprises:
calculating a viscosity quality control score according to the viscosity quality control characteristic; and
displaying the viscosity quality control information about the viscosity parameter by the viscosity quality control score.

In an embodiment, calculating a viscosity quality control score according to the viscosity quality control characteristic comprises:
performing weighted summation on each value characterizing the characteristic quantities to obtain the viscosity quality control score;
wherein, the value characterizing the characteristic quantities is an overlapping degree of the effective frequency range and the target frequency range when the viscosity quality control characteristic comprises the effective frequency range, or the value characterizing the characteristic quantities is a fitted line of the viscosity parameter and a degree of fit between data used for fitting when the viscosity quality control characteristic comprises the degree of matching, or the value characterizing the characteristic quantities is a proportion of continuous or discontinuous segments in the frequency dispersion curve when the viscosity quality control characteristic comprises a continuity of the frequency dispersion curve in the frequency dispersion distribution diagram, or the value characterizing the characteristic quantities is a signal-to-noise ratio of the frequency dispersion distribution diagram when the viscosity quality control characteristic comprises the signal-to-noise ratio of the frequency dispersion distribution diagram; or the value characterizing the characteristic quantities is a degree of influence of other pattern waves on a main shear wave when the viscosity quality control characteristic comprises different shear waves in multiple patterns in the frequency dispersion distribution diagram, the degree of influence of other pattern waves on a main shear wave comprises a proportion of energy of other pattern waves or main shear wave.

In an embodiment, displaying the viscosity quality control information about the viscosity parameter by the viscosity quality control score comprises:
generating a viscosity quality control distribution diagram of the region of interest according to the viscosity quality control score of each point in the region of interest; and
displaying the viscosity quality control distribution diagram.

In an embodiment, displaying the viscosity quality control information about the viscosity parameter according to the viscosity quality control characteristic comprises emptying a region in a viscosity parameter distribution diagram where the viscosity quality control information fails to meet a predetermined requirement according to the viscosity quality control characteristic; and the viscosity parameter distribution diagram is generated based on the viscosity parameter of each point in the region of interest.

According to a second aspect, a viscosity quality control method provided in an embodiment may include:
transmitting ultrasonic waves for detecting shear waves to a region of interest to obtain ultrasonic echo signals, the shear waves being propagating in the region of interest;
calculating a frequency dispersion distribution diagram according to the ultrasonic echo signals;
calculating a viscosity parameter according to the frequency dispersion distribution diagram; and
performing quality control on the viscosity parameter according to the frequency dispersion distribution diagram.

In an embodiment, performing quality control on the viscosity parameter according to the frequency dispersion distribution diagram comprises: performing quality control on the viscosity parameter by displaying the frequency dispersion distribution diagram.

In an embodiment, performing quality control on the viscosity parameter according to the frequency dispersion distribution diagram comprises:
obtaining a viscosity quality control characteristic of the viscosity parameter according to the frequency dispersion distribution diagram;
plotting the viscosity quality control characteristic on the frequency dispersion distribution diagram to generate a frequency dispersion characteristic graph; and
performing quality control on the viscosity parameter by displaying the frequency dispersion characteristic graph.

In an embodiment, performing quality control on the viscosity parameter according to the frequency dispersion distribution diagram comprises:
obtaining a viscosity quality control characteristic of the viscosity parameter according to the frequency dispersion distribution diagram; and
performing quality control on the viscosity parameter by displaying a value characterizing the viscosity quality control characteristic.

In an embodiment, performing quality control on the viscosity parameter according to the frequency dispersion distribution diagram comprises:
obtaining a viscosity quality control characteristic of the viscosity parameter according to the frequency dispersion distribution diagram;
calculating a viscosity quality control score according to the viscosity quality control characteristic; and
displaying viscosity quality control information about the viscosity parameter by the viscosity quality control score.

In an embodiment, performing quality control on the viscosity parameter according to the frequency dispersion distribution diagram comprises:
obtaining a viscosity quality control characteristic of the viscosity parameter according to the frequency dispersion distribution diagram; and
emptying a region in a viscosity parameter distribution diagram where the viscosity quality control information fails to meet a predetermined requirement according to the viscosity quality control characteristic, wherein the viscosity parameter distribution diagram is generated based on the viscosity parameter of each point in the region of interest.

In an embodiment, the viscosity quality control characteristic comprises any one or more of the following characteristic quantities:
an effective frequency range for calculating the viscosity parameter;
a degree of matching when performing model fitting for the viscosity parameter according to the frequency dispersion distribution diagram;
a continuity of a frequency dispersion curve in the frequency dispersion distribution diagram;
a signal-to-noise ratio of the frequency dispersion distribution diagram; and
different shear waves in multiple patterns in the frequency dispersion distribution diagram.

According to a third aspect, an ultrasonic imaging system provided in an embodiment may include:
an ultrasonic probe configured to transmit ultrasonic waves to a region of interest and receive corresponding ultrasonic echo signals;
a transmitting and receiving control circuit configured to control the ultrasonic probe to perform transmission of the ultrasonic waves and reception of the ultrasonic echo signals; and
a processor configured to perform the method according to any one embodiment of the present disclosure; and a display.

According to the ultrasonic imaging system and the viscosity quality control method mentioned in above embodiments, ultrasonic waves for detecting shear waves are transmitted to a region of interest in which the shear waves are propagated to obtain ultrasonic echo signals, a frequency dispersion distribution diagram is calculated based on the ultrasonic echo signals, and a viscosity parameter is calculated based on the frequency dispersion distribution diagram and is performed with quality control based on the frequency dispersion distribution diagram. Accordingly, the present disclosure provides a scheme of quality control on the viscosity parameter.

### BRIEF DESCRIPTION OF THE DRAWINGS

FIG. 1 is a schematically structural diagram of an ultrasonic imaging system according to an embodiment;

FIG. 2(a) is a schematic diagram of shear waves generated by strong focusing according to an embodiment, FIG. 2(b) is a schematic diagram of propagation of shear waves started from different locations by focusing on different regions respectively according to an embodiment;

FIG. 3(a) is a schematic diagram showing a relationship between shear-wave propagation time and distance in t-x domain according to an embodiment, FIG. 3(b) is a schematic diagram showing a relationship between shear-wave frequency and shear-wave number in f-v domain according to an embodiment, and FIG. 3(c) is a schematic diagram showing a relationship between shear-wave frequency and shear-wave propagation velocity in f-v domain according to an embodiment;

FIG. 4(a) is a schematic diagram of a relationship of shear waves in t-x domain according to an embodiment, FIG. 4(b) is a schematic diagram of a relationship of shear waves in f-k domain according to an embodiment, and FIG. 4(c) is a schematic diagram of a relationship of shear waves in f-v domain according to an embodiment;

FIG. 5(a), FIG. 5(b) and FIG. 5(c) are schematic diagrams of relationships of shear waves in f-v domain according to three embodiments;

FIG. 6(a) is a schematic diagram of an effective frequency range between 50 Hz and 300 Hz in an embodiment, and FIG. 6(b) is a schematic diagram of an effective frequency range between 50 Hz and 900 Hz in an embodiment;

FIG. 7 is a schematic diagram showing that the frequency dispersion curve of an embodiment does not change continuously with frequency;

FIG. 8 is a schematic diagram of a relative low signal-to-noise ratio of the frequency dispersion distribution diagram in an embodiment;

FIG. 9 is a schematic diagram showing a degree of matching using model fitting by a diagonal dashed line in an embodiment;

FIG. 10 is a schematic diagram of the frequency dispersion characteristic graph in an embodiment;

FIG. 11 is a schematic diagram of the frequency dispersion characteristic graph in another embodiment;

FIG. 12 is an example of displaying a frequency dispersion characteristic graph in the form of coordinate axes in an embodiment;

FIG. 13 is a schematic diagram of computing the viscosity quality control distribution diagram in an embodiment;

FIG. 14 is an example of displaying a B-mode image simultaneously with a frequency dispersion characteristic graph according to an embodiment;

FIG. 15 is an example of displaying a B-mode image, a viscosity parameter distribution diagram, and a frequency dispersion characteristic graph simultaneously according to an embodiment;

FIG. 16 is an example of textually prompting a viscosity quality control characteristic in an embodiment;

FIG. 17 is an example of illustrating a viscosity quality control score in a graphical manner in an embodiment;

FIG. 18 is an example of displaying a viscosity parameter distribution diagram and a viscosity quality control distribution diagram simultaneously in an embodiment;

FIG. 19 is a schematic diagram of a viscosity parameter shown as “XXX” in an embodiment;

FIG. 20 is a schematic diagram showing without a viscosity image in an embodiment;

FIG. 21 is a schematic diagram of emptying the viscosity image in an embodiment;

FIG. 22 is a schematic diagram of displaying a B-mode image, a viscosity image and a frequency dispersion characteristic graph simultaneously and textually prompting a value of a viscosity quality control characteristic and a viscosity quality control score on a display interface; and

FIG. 23 is a flowchart of the viscosity quality control method according to an embodiment.

### DETAILED DESCRIPTION

The present disclosure will be further described in detail below through specific embodiments with reference to the accompanying drawings. Common or similar elements are referenced with like or identical reference numerals in different embodiments. Many details described in the following embodiments are for better understanding the present disclosure. However, those skilled in the art can realize with minimal effort that some of these features can be omitted in different cases or be replaced by other elements, materials and methods. For clarity some operations related to the present disclosure are not shown or illustrated herein so as to prevent the core from being overwhelmed by excessive descriptions. For those skilled in the art, such operations are not necessary to be explained in detail, and they can fully understand the related operations according to the description in the specification and the general technical knowledge in the art.

In addition, the features, operations or characteristics described in the specification may be combined in any suitable manner to form various embodiments. At the same time, the steps or actions in the described method can also be sequentially changed or adjusted in a manner that can be apparent to those skilled in the art. Therefore, the various sequences in the specification and the drawings are only for the purpose of describing a particular embodiment, and are not intended to be an order of necessity, unless otherwise stated one of the sequences must be followed.

The serial numbers of components herein, such as “first”, “second”, etc., are only used to distinguish the described objects and do not have any order or technical meaning. The terms “connected”, “coupled” and the like here include direct and indirect connections (coupling) unless otherwise specified.

The inventors found that elasticity and viscosity may jointly affect the propagation velocity of shear waves in tissues, and viscosity may cause dispersion effect of shear waves in tissues, leading to different propagation conditions of shear waves with different frequencies in tissues. Based on this characteristic, when performing shear-wave elasticity imaging, the viscosity parameter can be calculated by extracting tissue motion information caused by shear-wave propagation related to the frequency dispersion effect of shear waves.

The inventors have found that in clinical practice, there are many factors that affect the accuracy and reliability of viscosity measurement, and these factors may cause deviation of the viscosity measurement, bringing great risks to clinical diagnosis. For example: the motion of tissues (caused by non-shear waves) may cause inaccurate shear wave measurement; additional dispersion effect may be introduced by structural features due to complexity of tissue structure, and shear waves in multiple patterns may thus also be generated, which may affect the calculation of dispersion curve; and the signal-to-noise ratio may become poor caused by poor contact between the ultrasonic probe and tissues, the attenuation of amplitude in the process of shear wave propagation, etc., causing the calculation error of shear wave dispersion to become larger. Therefore, it is important to perform quality control on viscosity measurement results to avoid misjudgment caused by other factors.

In consideration of the above factors affecting the viscosity measurement, the inventors propose some quality control characteristics for evaluating resulted viscosity parameter, thereby reflecting the accuracy and reliability of the calculated viscosity parameter through these quality control characteristics.

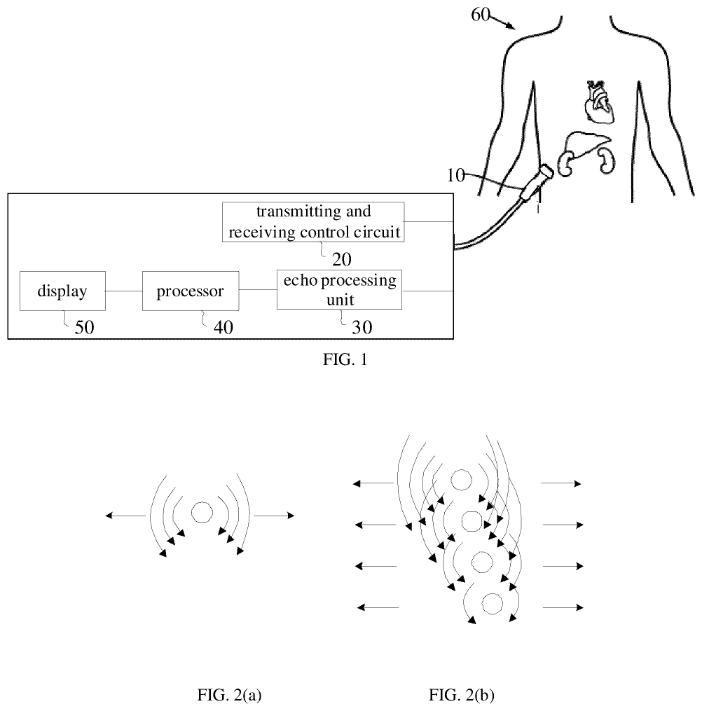

The present disclosure may be applied to an ultrasonic imaging system. Referring to FIG. 1, the ultrasonic imaging system in some embodiments may include an ultrasonic probe 10, a transmitting and receiving control circuit 20, an echo processing unit 30, a processor 40 and a display 50. These components are described below.

The ultrasonic probe 10 may be used to transmit ultrasonic waves to a region of interest and receive corresponding echo signals of the ultrasonic waves. In some embodiments, the ultrasonic probe 10 may be a matrix probe or a four-dimensional probe with mechanical device, which is not limited herein in this regard as long as the ultrasonic probe adopted can obtain the echo signals of the ultrasonic waves (or data) of a target region of a person being examined. In some specific embodiments, the ultrasonic probe 10 may include a plurality of array elements for conversion of electrical pulse signals and ultrasonic waves thereby performing the transmission of the ultrasonic waves to a biological tissue under examination 60 (a biological tissue of human or animal body) and the reception of ultrasonic echoes reflected from the tissue to obtain echo signals of the ultrasonic waves. The plurality of array elements included in the ultrasonic probe 10 may be arranged in a row to form a linear array, or in a two-dimensional matrix to form a plane array, or to form a convex array. The array elements may transmit ultrasonic waves according to excitation of electrical signals, or convert the received ultrasonic waves into electrical signals. Accordingly, each array element may be used to transmit ultrasonic waves to the region of interest of the biological tissue, or receive the echo signals of the ultrasonic waves returned from the tissue. When performing ultrasonic detection, a transmitting sequence and a receiving sequence may be used to control which array elements are used for transmitting ultrasonic waves and which array elements are used for receiving ultrasonic waves, or to control the array elements to transmit ultrasonic waves or receive ultrasonic echoes in a time-slot manner. All the array elements involved in transmission of the ultrasonic waves may be excited by electrical signals to simultaneously transmit the ultrasonic waves; alternatively, the array elements involved in transmission of the ultrasonic waves may be excited by several electrical signals with a certain time interval so as to continuously transmit the ultrasonic waves with a certain time interval.

In an example, the region of interest may be selected by users. For example, when a conventional ultrasonic image is displayed on the display 50, the region of interest may be selected on the conventional ultrasonic image. In another example, the position of the region of interest may be automatically determined by the processor 40 on a basic ultrasonic image based on an associated machine recognition algorithm. In yet another example, the region of interest may be obtained by semi-automatic detection. For example, the position of the region of interest may be automatically detected by the processor 40 on the basic ultrasonic image based on the machine recognition and then be modified or corrected by users to obtain a more accurate position of the region of interest.

The transmitting and receiving control circuit 20 may be used to control the ultrasonic probe 10 to perform the transmission of ultrasonic waves and the reception of the echo signals of the ultrasonic waves. Specifically, the transmitting and receiving control circuit 20 may be used to control the ultrasonic probe 10 transmit ultrasonic waves to the biological tissue such as the region of interest, and control the ultrasonic probe 10 to receive ultrasonic echoes of the ultrasonic waves returned by the tissue. In some embodiments, the transmitting and receiving control circuit 20 may be used to generate the transmitting sequence and the receiving sequence and output them to the ultrasonic probe 10. The transmitting sequence may be used to control part or all of the plurality of array elements in the ultrasonic probe 10 to transmit the ultrasonic waves to the biological tissue 60. Parameters of the transmitting sequence may include the number of array elements involved in transmission and transmitting parameters of the ultrasonic waves (such as amplitude, frequency, the number of waves transmitted, transmission interval, transmission angle, waveform and/or focus position). The receiving sequence may be used to control part or all of the plurality of array elements to receive echo waves of the ultrasonic waves from the tissue. Parameters of the receiving sequence may include the number of array elements involved in reception and receiving parameters of the echo waves (such as receiving angle, depth, etc.). The parameters of the transmitting sequence of the ultrasonic waves and the parameters of the receiving sequence of the echo waves may be different for different uses of the ultrasonic echo waves or different images generated based on the ultrasonic echo waves.

The echo processing unit 30 may be used to process the echo signals of the ultrasonic waves received by the ultrasonic probe 10, such as performing filtering, amplification, beamforming, etc. on the echo signals of the ultrasonic waves to obtain processed echo signals of the ultrasonic waves or data. In some embodiments, the echo processing unit 30 may output echo data of the ultrasonic waves to the processor 40. Alternatively, the echo data of the ultrasonic waves may be stored first in a memory and then be read out from the memory by the processor 40 when an operation based on the echo data of the ultrasonic waves is required. It should be appreciated by those skilled in the art that the echo processing unit 30 may be omitted when it is unnecessary to filter, amplify and beamform the echo signals of the ultrasonic waves in some embodiments.

The processor 40 may be used to obtain the echo signals of the ultrasonic waves or data and obtain required parameters or images by using a related algorithm. The processor 40 in some embodiments of the present disclosure may include but not limit to a central processing unit (CPU), a micro controller unit (MCU), a field-programmable gate array (FPGA) and digital signal processing (DSP) devices used to interpret computer instructions and process data in computer software. In some embodiments, the processor 40 may be configured to execute computer applications in non-transitory computer-readable storage medium to enable a sample analysis device to perform a corresponding detection procedure.

The display 50 may be used to display information, such as parameters and images obtained by the processor. It should be appreciated by those skilled in the art that the ultrasonic imaging system itself may not integrate a display unit, but instead may be coupled to a computer device (e.g. a computer) to display information via the display unit of the computer device in some embodiments.

Above are some descriptions of the ultrasonic imaging system. In some embodiments, with the ultrasonic imaging system, shear waves propagated in the region of interest may be detected to calculate the elasticity parameter and/or viscosity parameter of the region of interest.

There are various ways to generate shear waves in the region of interest. For example, the shear waves may be generated in the region of interest by external vibration. For another example, the shear waves may be generated in the region of interest by transmitting special pulses (such as acoustic radiation force impulses, ARFIs) to the region of interest by the ultrasonic probe 10. When the shear waves are generated in the region of interest by the acoustic radiation force impulses, the acoustic radiation force impulses may or may not be focused. Specifically, when the acoustic radiation force impulses are strongly focused, wave source generating the shear waves may be more focused; and when the acoustic radiation force impulses are weakly focused, the range of the shear waves generated is wider, and multiple shear-wave point sources may be approximated as propagating from multiple starting points in the range of shear waves generated. In addition, the range may also be widened directly by generating the shear waves at a plurality of different positions. Taking the acoustic radiation force impulses as an example, the propagation of the shear waves starting from the different positions may be generated by transmitting the acoustic radiation force impulses multiple times and focusing on different regions respectively. For example, FIG. 2(a) shows the shear waves generated by strong focusing, and FIG. 2(b) shows the propagation of the shear waves starting from the different positions generated by focusing on different regions. Of course, a larger range of shear waves can also be generated by external vibration, for example, by applying vibration at different positions from which the propagation of the shear waves can be generated.

In some embodiments of the present disclosure, the ultrasound probe 10 may emit ultrasonic waves for detecting shear waves to the region of interest obtain ultrasonic echo signals, the shear waves propagate in the region of interest; the processor 40 may calculate the frequency dispersion distribution diagram according to the ultrasonic echo signals; the processor 40 may calculate the viscosity parameter according to the ultrasonic echo signals, for example, firstly calculating the frequency dispersion distribution diagram, and then calculating the viscosity parameter based on the frequency dispersion distribution diagram; and the processor 40 may perform quality control on the calculated viscosity parameter according to the frequency dispersion distribution diagram.

The frequency dispersion distribution diagram may also be referred to as a frequency dispersion curve distribution diagram or a frequency dispersion curve graph. A description is first given to the calculation of the frequency dispersion distribution diagram.

According to wave characteristics, when the shear waves propagate through a certain position in the tissue, the tissue at the corresponding position may vibrate, and when the shear waves propagate away from a certain position, the tissue at this position may revert to original conditions. Therefore, the vibration of the tissue caused by the shear waves may be observed by transmitting ultrasonic waves to the tissue, and the motion information of the tissue over a period of time can be obtained by correlation and comparison of the ultrasonic waves echo signals obtained at different times. The motion information may be a displacement of the tissue relative to a reference moment, the motion velocity of the tissue, the motion acceleration of the tissue, the strain of the tissue, etc. or data after further processing by filtering, differentiating, integrating, etc. based on the above variables. The correlation and comparison may be a comparison calculation between ultrasonic echo signals obtained at different adjacent moments, or a comparison calculation between ultrasonic echoes at different moments and echo signals at the same reference moment. The algorithm for the correlation and comparison may comprise a general algorithm for conventional tissue displacement detection, such as a block-matching based cross-correlation comparison algorithm, a Doppler shift based calculation method, a phase shift detection based method, etc. The embodiments of the present disclosure do not limit the specific algorithm used to detect tissue motion information.

Therefore, after the ultrasonic probe 10 transmits ultrasonic waves for detecting shear waves to the region of interest and the processor acquires corresponding ultrasonic echo signals from the ultrasonic probe 10, a time-space distribution diagram of the shear waves propagating in the region of interest (time-distance domain, a relationship between the propagation position of the shear waves and time) can be obtained according to the ultrasonic echo signals, and then the time-space distribution diagram of the shear waves propagating in the region of interest is transformed into a frequency-wave number domain or a frequency-velocity domain to obtain the frequency dispersion distribution diagram of the shear waves, namely, the relationship between the propagation velocity of the shear waves and the frequency, wherein the wave number of the shear waves is the inverse of the wavelength of the shear waves.

In some embodiments, methods such as the 2DFF transform, the Tau-p transform, the Radon transform, the E-V decomposition, or other transform methods derived therefrom may be employed to convert the time-space distribution diagram of the shear waves as they propagate in the region of interest to shear waves, in order to obtain a relationship between frequency and velocity or velocity-related quantities (e.g. slowness, wave number, etc.).

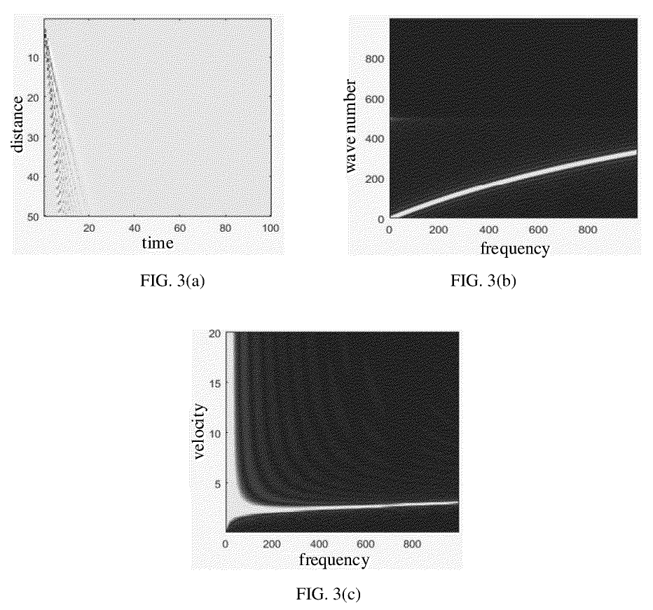

Taking 2DFFT as an example, and a specific process thereof may include that: the 2DFF transform method transforms the propagation of the shear waves from the t-x space (i.e. the relationship of the propagation position of the shear waves with time) shown in FIG. 3(a) to the f-k space (i.e. the relationship of the frequency of the shear wave propagation with the wave number of the shear waves) shown in FIG. 3(b). It can be clearly seen from the t-x space of shear-wave propagation that the positions of the peaks and troughs of the shear waves at different moments may represent the propagation process of wave group of the shear waves. It can be seen from the f-k space of the shear waves that the wave number distribution corresponding to different frequencies, wherein the larger the value in the f-k domain, the higher the energy. The f-k domain has required information to extract shear wave dispersion, but the display is not intuitive enough. The relationship among velocity and wave number and frequency is v=f/k, and it can continue to transform from the f-k domain of shear waves to the f-v domain of shear waves, which represents the relationship between the frequency of shear waves and the propagation velocity of shear waves. FIG. 3(c) is an example of the distribution of shear waves in the f-v domain. The shear wave which is maximum in for example energy here is referred to as a main shear wave (or a main pattern wave; and correspondingly, the shear waves which are not maximum in for example energy are referred to as other pattern waves, and the value thereof is maximum in f-k domain and in f-v domain, so the position where each frequency maximum value is located in the frequency dispersion distribution diagram is extracted to obtain the frequency dispersion curve for calculating viscosity parameter.

Tau-p transform, radon transform or other transform methods derived from them may be used to calculate the frequency dispersion distribution of shear waves. The general idea thereof is similar, in which the propagation of the shear waves is transformed from t-x domain to other spatial domain and then to f-v domain. Tau-p transform may transform shear wave propagation from the t-x domain to intercept-slowness domain, wherein the intercept is transformed to frequency-slowness domain after Fourier transform, and the slowness is the derivative of velocity, which can be converted to the frequency-velocity domain.

The frequency dispersion distribution diagram described herein may include a relationship between the frequency of shear wave propagation and the wave number of shear waves, or between the frequency of shear wave propagation and its velocity. FIG. 3(b) and FIG. 3 (c) are examples of the frequency dispersion distribution diagram.

These are some description of the frequency dispersion distribution diagram.

In some embodiments, the processor 40 may compute the viscosity parameter based on the frequency dispersion distribution diagram. For example, a slope of the frequency dispersion curve in dispersion distribution diagram with respect to shear wave frequency and shear wave propagation velocity is calculated as the viscosity parameter or one of the viscosity parameter. For another example, the viscosity parameter is calculated according to phase velocities of shear waves of at least two different frequencies in the frequency dispersion distribution diagram. For yet another example, the viscosity parameter is calculated according to phase velocities of shear waves of at least two different frequencies in the frequency dispersion distribution diagram and corresponding frequencies. Since the shear-wave source has wider frequency band information, the shear wave propagation velocity calculated from original shear wave signals is an integrated propagation velocity of shear waves of various frequencies, which is referred to as a shear wave group velocity; and the propagation velocity of the shear waves calculated using separated shear wave components is the propagation velocity of the shear waves at a corresponding frequency, which thus is referred to as the phase velocity of the shear waves. The difference or ratio of the phase velocity of shear waves of different frequencies, or the slope of the phase velocity with respect to the shear wave frequency for the shear wave of different frequencies may be taken as the viscosity parameter.

In some embodiments, the processor 40 may perform quality control on the calculated viscosity parameter according to the frequency dispersion distribution diagram.

For example, the viscosity parameter may be performed with quality control by displaying the frequency dispersion distribution diagram directly, by the frequency dispersion characteristic graph, by the viscosity quality control characteristic, or by the viscosity quality control score; and it can be understood that the viscosity parameter may be performed with quality control by the combination of the aforesaid ways.

In some embodiments, the processor 40 may obtain the viscosity quality control characteristic of the viscosity parameter according to the frequency dispersion distribution diagram, and perform quality control on the viscosity parameter by the viscosity quality control characteristic. In some examples, the viscosity quality control characteristic may include one or more of the following characteristic quantities:
an effective frequency range for calculating the viscosity parameter;
a degree of matching when performing model fitting for the viscosity parameter according to the frequency dispersion distribution diagram;
a continuity of the frequency dispersion curve in the frequency dispersion distribution diagram;
a signal-to-noise ratio of the frequency dispersion distribution diagram; and
different shear waves in multiple patterns in the frequency dispersion distribution diagram.

These viscosity quality control characteristics or characteristic quantities are explained one by one below.

(1) Shear Waves in Multiple Patterns

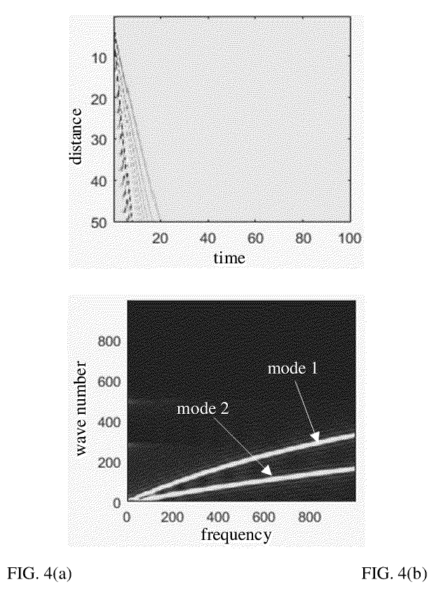

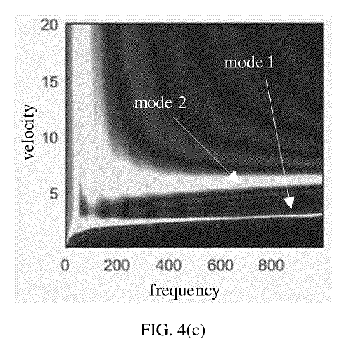

The actual structures for tissues are complex, and the tissues themselves may have structural features such as stratification, blood vessels and fibers, leading to generating different shear waves in multiple patterns; i.e. the shear waves may, due to reflection when they encounter such structural features in the tissues during propagation in the tissues, produce the different shear waves in multiple patterns (they may be different in for example frequency, propagation velocity, etc.). Waves in different patterns may have different propagation velocities, which may directly affect the calculation of dispersion curve. Waves in different patterns are superimposed together in the t-x domain and cannot be recognized, but they can be transformed to other spatial domains for display, thereby the multiple modes of waves can be recognized in the f-v domain and the f-k domain, as shown in FIG. 4(a), FIG. 4(b) and FIG. 4(c). FIG. 4(a), FIG. 4(b) and FIG. 4(c) are schematic diagrams of shear waves in t-x, f-k and f-v domains. This feature can be used to judge the quality of viscosity measurement, or can be used to perform quality control on the calculated viscosity parameter. For example, the appearance of the shear waves in multiple patterns may indicate that the structural characteristics of the measured region are obvious, and the reliability of viscosity measurement is low.

In an embodiment, when the viscosity parameter is quality controlled by the characteristic quantities i.e. different shear waves in multiple patterns in the frequency dispersion distribution diagram, the recognized waves of multiple modes may be prompted to visually prompt users for the presence of the shear waves in multiple patterns.

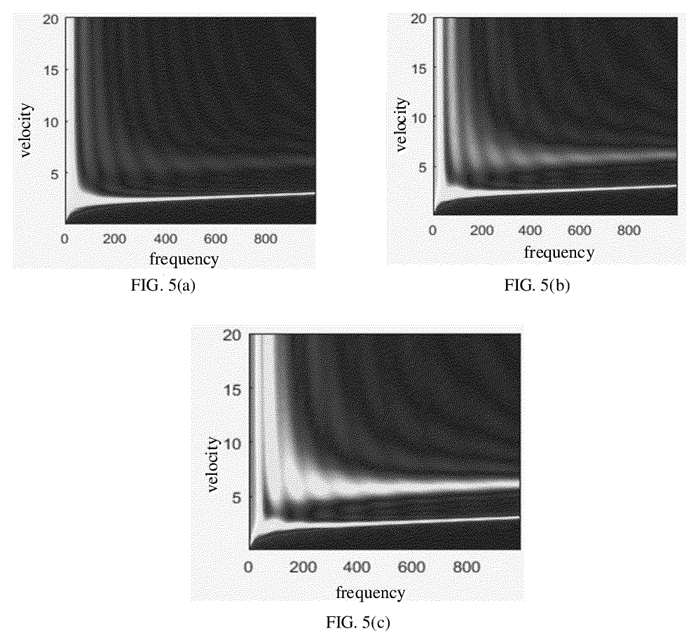

In an embodiment, the influence of other pattern waves on the main shear wave may be quantified. For example, a relationship of energy between other pattern waves and the main shear wave. The smaller the energy ratio of other pattern waves to the main shear wave, or the larger the total energy ratio of the main shear wave, the smaller the influence of tissue structural characteristics on the viscosity measurement. From the figures (a) to (c), the waves of other modes are more and more obvious, and the influence of structure is more and more significant. For example, the energy ratio of other pattern waves to the main shear wave is used to quantify the influence of other pattern waves, as shown in FIG. 5(a), FIG. 5(b) and FIG. 5(c), the frequency from 400 to 800 is calculated, the energy ratios of other pattern waves to the main shear wave are 0.2, 0.5 and 1, respectively. This indicates that the other pattern waves are more and more obvious, the effect of tissue structure on viscosity measurement is becoming greater, and the reliability of calculated viscosity parameter is less and less.

(2) Effective Frequency Range

In the viscosity measurement, it is necessary to calculate the viscosity parameter according to the propagation velocity of shear waves with different frequencies. In an example of selecting a shear wave with a lower frequency, the shear wave is greatly influenced by the structure due to its low frequency and long wavelength. In some examples of selecting a shear wave with a higher frequency, the calculation of velocity is large in error due to the fast attenuation, low amplitude and signal-to-noise ratio difference of the high-frequency shear wave. In addition, in a case where the shear waves are sheared by means of acoustic radiation force, the frequency component of the shear waves is also related to the nature of the acoustic radiation force generated by the excitation on the tissue by the ultrasonic pulse, and the amplitude, waveform and focus size of the acoustic pulses may limit the frequency component of the shear waves. Therefore, the selection of frequency range directly affects the quality of the viscosity measurement. The frequency range of the signal cannot be observed in the t-x domain, but after changing to the f-v domain, the available frequency range can be clearly visible, and it can be directly determined whether the actual signal frequency components meet the requirements.

In some embodiments, the effective frequency range and/or the target frequency range may be marked on the frequency dispersion distribution diagram.

In some embodiments, the overlapping degree of the effective frequency range and the target frequency range may be calculated.

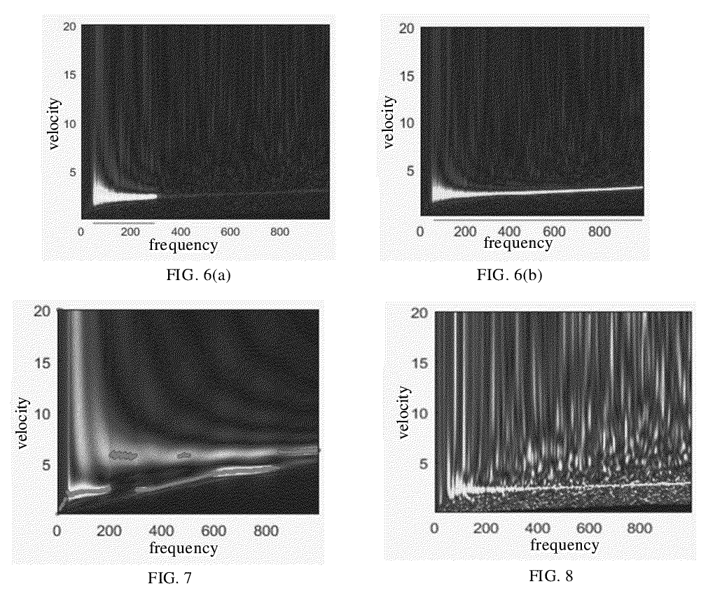

Herein, the effective frequency range may refer to a range of frequencies of the shear waves that can used to calculate the viscosity parameter on a frequency distribution map. When the shear waves with the range of frequencies are used to calculate the viscosity parameter, the result thereof is relatively accurate, that is, the viscosity is of good quality; which can be observed by the sharpness of the frequency dispersion curve in the frequency distribution map, such as 50 Hz to 300 Hz for the effective frequency range in FIG. 6(a) and 50 Hz to 900 Hz for the effective frequency range in FIG. 6(b). The target frequency range is the range actually used for calculating the viscosity parameter. Generally, a default range for calculating viscosity parameter may be preset in the system, which is referred to as the target frequency range.

Assuming that target frequency range is 100 to 400 Hz, the degree of overlap in FIG. 6(a) is 0.66, and the degree of overlap in FIG. 6(b) is 1, it can be seen that the greater the degree of overlap, the better the quality of the viscosity parameter calculated by shear waves of the target frequency range.

(3) Continuity of the Frequency Dispersion Curve in the Frequency Dispersion Distribution Diagram or the Accuracy of Calculation of Position

After obtaining the frequency dispersion distribution diagram, the position of the main shear wave may be determined by locating a position where the maximum value of the frequency dispersion distribution is located, and the frequency dispersion curve of the main shear wave may be extracted. However, there may be a lot of interference in actual operation, resulting in a deviation of the location of the main shear wave. For example, when the main mode is extracted correctly, the frequency dispersion curve varies continuously with frequency without any jumping data points. As shown in FIG. 7, there is an example where the frequency dispersion curve does not change continuously with frequency, in which a large number of jumping data points occur. The more accurately the location of the main shear wave is calculated, i.e. the more continuous the frequency curve, the better the quality of the calculated viscosity parameter.

In some embodiments, In some embodiments, the accuracy of the position calculation may be determined by the continuity of the extracted dispersion curve. For example, the continuity judgement method of a curve can judge a jump data point by comparing a threshold value with the previous data or previous data; or it may be measured through data difference, gradient, data variance and normalized variance.

(4) Signal-to-Noise Ratio

In actual operation, the low amplitude of shear wave and poor signal-to-noise ratio may be caused by the poor contact between ultrasonic probe and tissues, too deep probing depth, small acoustic radiation force generated by excitation, or large attenuation coefficient of shear waves. In the frequency dispersion distribution diagram, the background is not smooth and the signal is inseparable from the background. In this case, the frequency dispersion curve extracted has a large error and low credibility. FIG. 8 is an example.

In some embodiments, the quality of the calculated viscosity parameter may be evaluated by the signal-to-noise ratio of the frequency dispersion distribution diagram; and the higher the signal-to-noise ratio, the higher the quality of the viscosity parameter.

(5) Degree of Matching for Model Fitting

After extracting the frequency dispersion curve of the shear waves, the viscosity parameter is calculated by model fitting. The higher the degree of matching for model fitting, the more accurate the calculated viscosity parameter, i.e. the better the quality.

In some embodiments, the frequency dispersion curve represented by the calculated viscosity parameter is prompted in the frequency dispersion distribution diagram for users to determine the degree of matching between the calculated curve and the actual data, for example, the diagonal dotted line in FIG. 9 shows the frequency dispersion curve represented by the calculated viscosity parameter.

In some embodiments, it is also possible to quantify the degree of matching between the frequency dispersion curve represented by the obtained viscosity parameter (i.e. the curve obtained by fitting the actual data) and the actual data. For example, the quantization may be performed by calculating the correlation coefficient or residual between the fitted curve and the actual data, the larger the correlation coefficient or the smaller the residual, the higher the degree of matching.

The above are some description of the viscosity quality control characteristic.

In some examples, the viscosity parameter may be performed with quality control by showing the viscosity quality control characteristic qualitatively or quantitatively, which is explained below.

In some embodiments, the processor 40 may draw a viscosity quality control characteristic on the frequency dispersion distribution diagram to generate a frequency dispersion characteristic graph; and the processor 40 may quality control the viscosity parameter by controlling the display 50 to display the frequency dispersion characteristic graph. The frequency dispersion characteristic graph may be generated by extracting, marking or enhancing the viscosity quality control characteristic in the frequency dispersion distribution diagram, or weakening the display background. For example, when the viscosity quality control characteristic includes the effective frequency range, the effective frequency range may be marked on the frequency dispersion distribution diagram, or the effective frequency range and the target frequency range for calculating the viscosity parameter may be marked on dispersion distribution diagram. For another example, when the viscosity quality control characteristic includes the degree of matching, the fitted line of the viscosity parameter may be obtained and plotted on the frequency dispersion distribution diagram, e.g. the fitted line is plotted on the frequency dispersion distribution diagram in dashed lines. For yet another example, when the viscosity quality control characteristic includes the continuity of the frequency dispersion curve in the frequency dispersion distribution diagram, only consecutive points in the frequency dispersion curve are connected on the frequency dispersion distribution diagram to draw the frequency dispersion curve, so that users can intuitively judge the continuity of the frequency dispersion curve. For still yet another example, when the viscosity quality control characteristic includes different shear waves in multiple patterns in the frequency dispersion distribution diagram, the frequency dispersion curve for each shear wave is extracted and plotted on the frequency dispersion distribution diagram so that users can see how many waves there has.

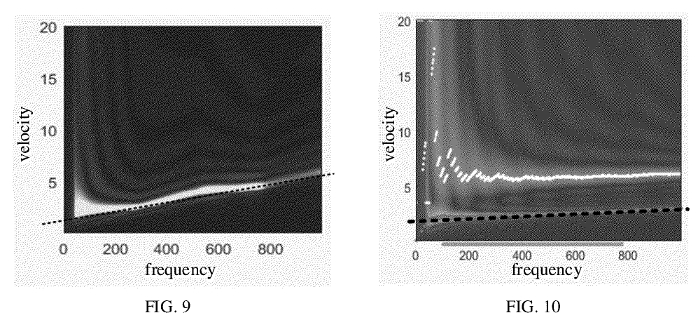

FIG. 10 is an example of quality control of the viscosity parameter by the frequency dispersion characteristic graph, in which the effective frequency range, degree of matching, different shear waves in multiple patterns in the frequency dispersion distribution diagram, etc. may be plotted on the frequency dispersion distribution diagram to generate the frequency dispersion characteristic graph.

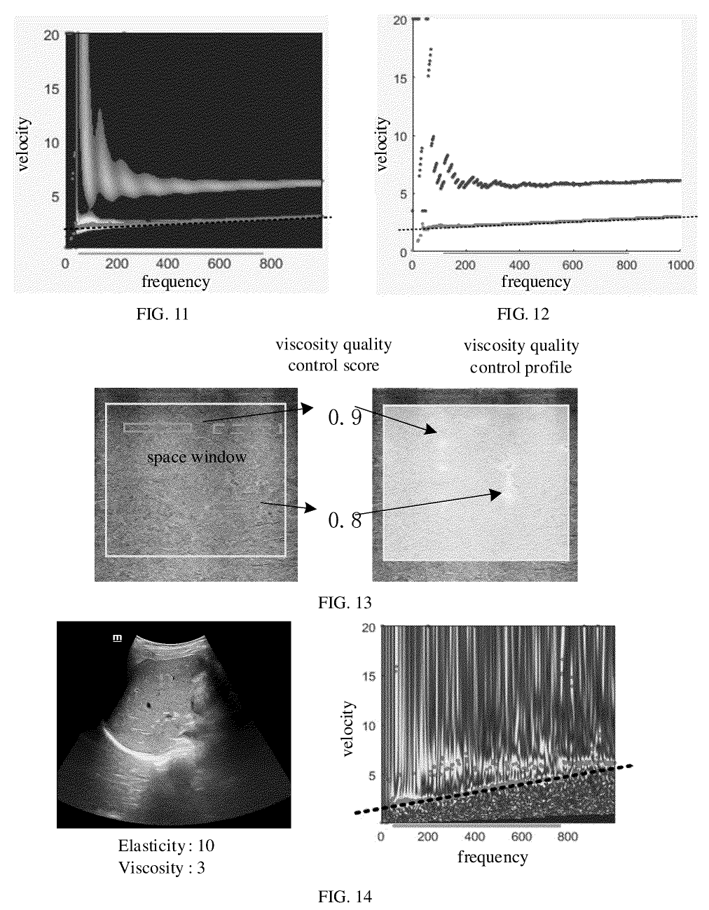

In some embodiments, the processor 40 may, before drawing the viscosity quality control characteristic on the frequency dispersion distribution diagram, perform foreground feature enhancement or background fading on the frequency dispersion distribution diagram. For example, the background of the frequency dispersion distribution diagram is pre-configured, such as weakening the background image, strengthening the frequency dispersion curve of shear waves under each transmission mode, extracting the frequency dispersion curve of each shear wave, and displaying the frequency dispersion characteristic graph in the form of coordinate axis, etc. FIG. 11 is an example in which before drawing the frequency dispersion characteristic graph on the frequency dispersion distribution diagram, the background of the frequency dispersion distribution diagram is weakened by thresholding to obtain the frequency dispersion characteristic graph. FIG. 12 is an example of displaying frequency dispersion characteristic graph in the form of coordinate axes.

In some embodiments, the processor 40 may display the viscosity quality control information about the viscosity parameter by the value characterizing the viscosity quality control characteristic, or perform quality control on the viscosity parameter by controlling the display 50 to display a value of the viscosity quality control characteristic. The value of the viscosity quality control characteristic is a value obtained after quantization of the viscosity quality control characteristic, such as the number of wave modes, the ratio of main mode to total energy, an effective frequency range, accuracy of position calculation, a signal-to-noise ratio, a fitting correlation coefficient, and so on. The value obtained after quantization of the viscosity quality control characteristic may be a continuous value quantized, or may be classified, for example, the ratio of the main mode to the total energy may be 0-100%, or may be classified into low, medium and high.

For example, when the viscosity quality control characteristic includes the effective frequency range, the value of the effective frequency range may be displayed, or the value of the effective frequency range and the value of the target frequency range used for calculating the viscosity parameter may be displayed, or the overlapping degree of the effective frequency range and the target frequency range may be calculated and displayed. For another example, when the viscosity quality control characteristic includes the degree of matching, the fitted line of the viscosity parameter and the degree of fit between data used for fitting may be calculated and the degree of fit may be displayed. The degree of fit may include an average absolute difference value, a mean square error, a root mean square error, a coefficient of determination R2 or a correlation coefficient. For yet another example, when the viscosity quality control characteristic includes the continuity of the frequency dispersion curve in the frequency dispersion distribution diagram, the proportion of continuous or discontinuous segments in the frequency dispersion curve may be calculated and displayed. For still another example, when the viscosity quality control characteristic includes the signal-to-noise ratio of the frequency dispersion distribution diagram, the signal-to-noise ratio of the frequency dispersion distribution diagram may be calculated and displayed. For yet still another example, when the viscosity quality control characteristic includes the different shear waves in multiple patterns in the frequency dispersion distribution diagram, the number of different shear waves in multiple patterns in the frequency dispersion distribution diagram may be calculated and displayed, or the main shear wave may be determined and a degree of influence of other pattern waves on the main shear wave may be calculated and displayed. The degree of influence of other pattern waves on a main shear wave comprises a proportion of energy of other pattern waves or main shear wave.

In an embodiments, the processor 40 may calculate a viscosity quality control score according to the viscosity quality control characteristic, and display the viscosity quality control information about the viscosity parameter by the viscosity quality control score. In some embodiments, the processor 40 may calculate the viscosity quality control score according to the viscosity quality control characteristic as follows: performing weighted summation on each value characterizing the characteristic quantities to obtain the viscosity quality control score. In some embodiments, when the viscosity quality control characteristic includes the effective frequency range, the value characterizing the characteristic quantities may be the overlapping degree of the effective frequency range and the target frequency range; when the viscosity quality control characteristic includes the degree of matching, the value characterizing the characteristic quantities may be the fitted line of the viscosity parameter and the degree of fit between data used for fitting; when the viscosity quality control characteristic includes the continuity of the frequency dispersion curve in the frequency dispersion distribution diagram, the value characterizing the characteristic quantities may be the proportion of continuous or discontinuous segments in the frequency dispersion curve; when the viscosity quality control characteristic includes the signal-to-noise ratio of the frequency dispersion distribution diagram, the value characterizing the characteristic quantities may be the signal-to-noise ratio of the frequency dispersion distribution diagram; and when the viscosity quality control characteristic includes the different shear waves in multiple patterns in the frequency dispersion distribution diagram, the value characterizing the characteristic quantities may be the degree of influence of other pattern waves on the main shear wave. The degree of influence of other pattern waves on the main shear wave may include the proportion of energy of other pattern waves or the main shear wave.

It can be seen that viscosity quality control score can combine one or more viscosity quality control characteristic to perform the weighting calculation.

For example, the viscosity quality control score QF=w1*a1+w2*a2+w3*a3+w4*a4+w5*a5 . . .

where a1 is the influence of other pattern waves, a2 is the effective frequency range, a3 is the accuracy of position calculation or the continuity of the frequency dispersion curve in the frequency dispersion distribution diagram, a4 is the signal-to-noise ratio, a5 is the degree of matching for model fitting, and w1, w2, w3, w4 and w5 are weight coefficients of each viscosity quality control characteristic. It is obvious that the greater the weight is, the more influence the relative viscosity quality control characteristic can have on the final viscosity measurement results. When the weight is 0, the influence of the viscosity quality control characteristic may be ignored. For example, when the computational theory model for the viscosity parameter is a multi-layer structure, the frequency dispersion data of the main mode and other modes may be used, and in this case, the waves of other modes should be used as signals rather than interference, and the weight coefficient of w1 should be low. In addition, the viscosity quality control score may take full account of the quality of B-mode images and the characteristics of shear-wave time conventional B-mode image quality information, and conventional quality information about shear wave elasticity imaging.

In some embodiments, the processor 40 may display the viscosity quality control information about the viscosity parameter by the viscosity quality control score as follows: the processor 40 may generate the viscosity quality control distribution diagram of the region of interest according to the viscosity quality control score of each point in the region of interest; and the processor 40 may control the display 50 to display the viscosity quality control distribution diagram.

With reference to FIG. 13, the frequency dispersion distribution can be calculated on each point in the space and its surrounding space window, and after quantizing the features of the frequency dispersion distribution, each viscosity quality control characteristic may be combined, or various information such as B-mode image quality and shear-wave time domain features may be combined, thereby obtaining the viscosity quality control score; and each point may be traversed in the space to obtain the viscosity quality control score, and the quality control scores of each point are combined together to form the distribution of quality control distribution to obtain the viscosity quality control distribution diagram.

In some embodiments, the processor 50 may empty a region of the viscosity parameter distribution diagram where the viscosity quality control information fails to meet a predetermined requirement, the viscosity parameter distribution diagram may be generated based on the viscosity parameter of each point in the region of interest. For example, each value characterizing the characteristic quantities may be weighted and summed to obtain the viscosity quality control score, and the viscosity quality control distribution diagram of the region of interest may be generated according to the viscosity quality control score of each point in the region of interest.

In some embodiments, the processor 40 may perform quality control on the viscosity parameter by controlling the display 50 to display the frequency dispersion distribution diagram. In some instances, the frequency dispersion distribution diagram may represent the relationship between shear wave propagation properties and frequency, such as the relationship between shear wave propagation velocity and frequency, with the horizontal coordinate being frequency and the vertical coordinate being velocity, it being understood that the horizontal and vertical coordinates may be substituted for each other. Alternatively, the shear wave velocity and frequency may be calculated to obtain the slowness (inverse of velocity) versus frequency or wave number (ratio of velocity to frequency) versus frequency as the frequency dispersion distribution diagram.

In some embodiments, the processor 40 may control the display 50 to display one or more of the frequency dispersion distribution diagram, the frequency dispersion characteristic graph, the viscosity quality control characteristic, the viscosity quality control score, and the viscosity quality control distribution diagram in combination with any one or more of a conventional B-mode image, an elasticity image, an elasticity quality control distribution diagram, or the viscosity parameter distribution diagram (which may also be referred to as a viscosity image) for a user to comprehensively judge the quality of the imaging. The elasticity image may be generated according to elasticity parameters of each point in the region of interest. The elasticity parameters may also include various types according to different elasticity imaging methods; for example, the elasticity parameters may be a strain, a strain ratio and a strain rate for conventional pressed type elasticity imaging, or be a shear wave velocity, a shear wave velocity ratio, a Young's modulus, a shear modulus, a Young's modulus ratio, a shear modulus ratio and a shear wave propagation distance for shear wave elasticity imaging. In this embodiment, the parameters may include, but be not limited to: strain-to-strain ratio, strain-time curve, shear wave velocity-to-shear wave velocity ratio, elastic modulus-to-elastic modulus ratio, elastic histogram statistics, and so on. An elasticity quality control distribution diagram is a graph used to quality control an elasticity image.

Some embodiments may be: simultaneously displaying B-mode images, and the frequency dispersion characteristic graph or the frequency dispersion distribution diagram; and FIG. 14 is an example.

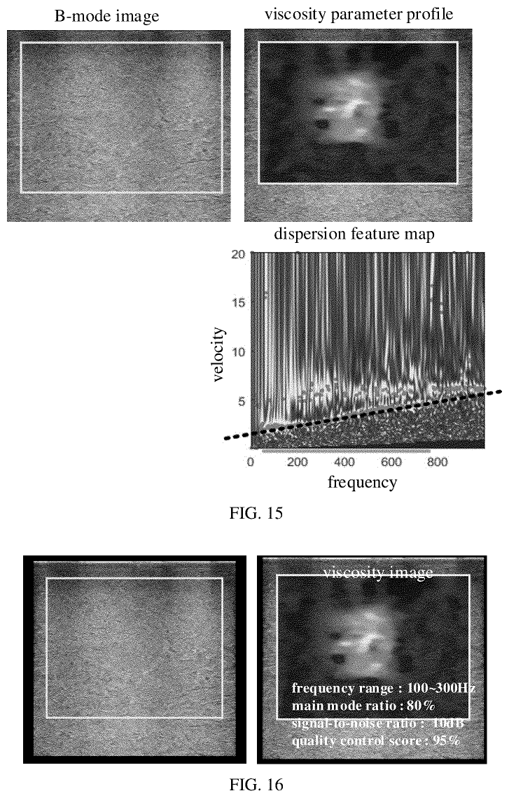

Some embodiments may be: simultaneously displaying B-mode images (in which the region of interest may be marked), and the frequency dispersion characteristic graph or the frequency dispersion distribution diagram; and the viscosity quality control characteristic or the viscosity quality control score may be prompted on a display interface. Of course the B-mode image (in which the region of interest may be marked), the viscosity parameter distribution diagram and the frequency dispersion characteristic graph may be displayed simultaneously, and FIG. 15 is an example.

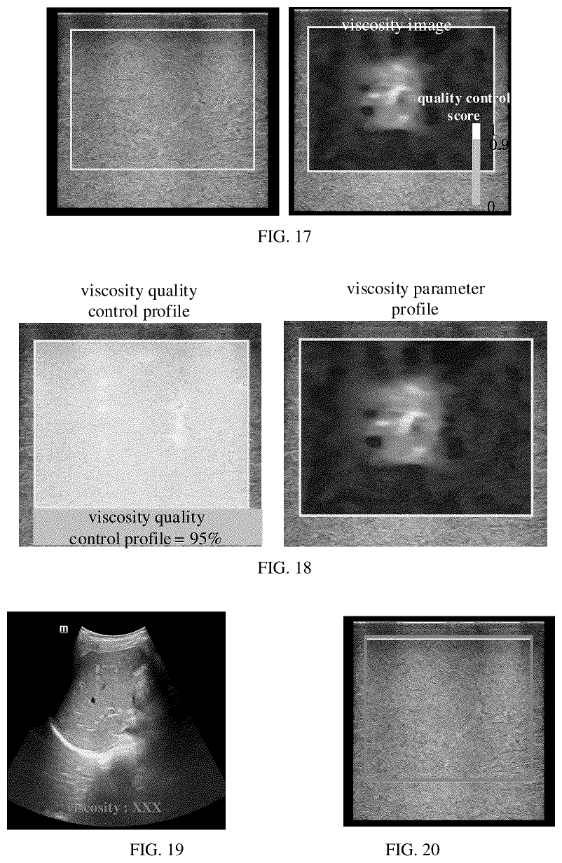

Some embodiments may be: prompting the viscosity quality control characteristic or the viscosity quality control score, such as the number of wave modes, the proportion of energy of the main shear wave, the effective frequency range, the signal-to-noise ratio, the continuity of the frequency dispersion curve, and the correlation coefficient of model fitting, on the display interface in a textual or graphical manner, or one or more of these parameters may be displayed, or the viscosity quality control score resulting from combining these parameters may be displayed. The value of the viscosity quality control characteristic may be displayed at any position of the display interface. The color of the text and graphics of the viscosity quality control characteristic and the viscosity quality control score can vary with the value, such as red when the viscosity quality control score is less than 0.5, yellow between 0.5 and 0.8, and green when the viscosity quality control score is greater than 0.8. The prompted graphics of the viscosity quality control characteristic and the viscosity quality control score may be in other forms such as a bar or a pie chart. FIG. 16 is an example in which the viscosity quality control characteristic is prompted textually. FIG. 17 is yet another example, in which the viscosity quality control score is shown graphically (as a bar), and the viscosity quality control score is shown as 0.9.

Some embodiments may be: calculating the viscosity quality control score of each position in the region of interest to obtain the viscosity quality control distribution diagram, and displaying the viscosity parameter distribution diagram simultaneously. FIG. 18 is an example thereof.

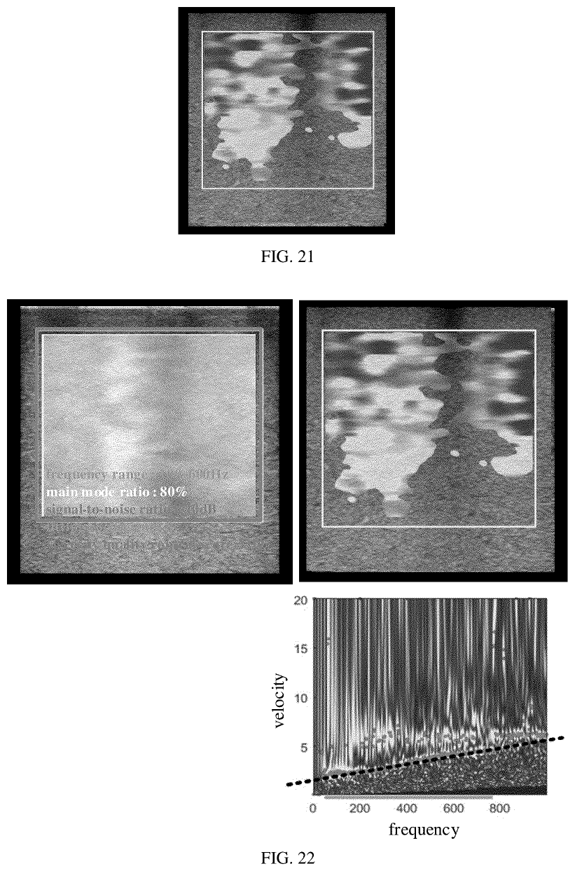

Some embodiments may be: not displaying the viscosity parameter or displaying it as error when the viscosity quality control characteristic fails to meet the requirements according to the viscosity quality control characteristic. For example, the viscosity parameter is displayed as XXX, or the viscosity image is not displayed; or the viscosity image is emptied according to the viscosity quality control distribution diagram, such as emptying a region of the viscosity parameter distribution diagram where the viscosity quality control information fails to meet a preset requirement. FIG. 19, FIG. 20 and FIG. 21 are three examples, in which FIG. 19 shows an example of the viscosity parameter displayed as XXX, FIG. 20 shows an example of without displaying the viscosity image, and FIG. 21 show an example of the viscosity image being emptied.

It may be appreciated that several of the above prompts may also be combined, such as simultaneously displaying a B-mode image, a viscosity image and a frequency dispersion characteristic graph, and textually prompting the value of the viscosity quality control characteristic and the viscosity quality control score on a display interface, the color of the text varying with the size of the value. For example, FIG. 22 is an example thereof.

A viscosity quality control method is also provided according some embodiments of the present disclosure, which is explained below.

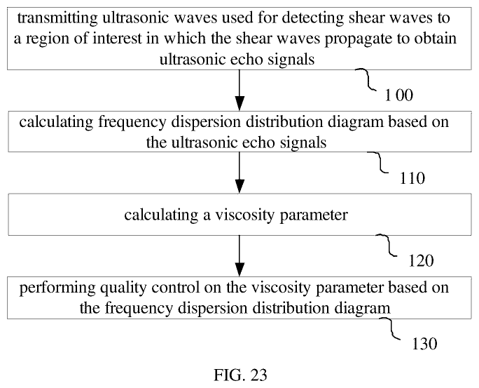

Referring to FIG. 23, the viscosity quality control method in some embodiments may include the following steps:

Step 100: transmitting ultrasonic waves used for detecting shear waves to a region of interest to obtain ultrasonic echo signals, the shear waves being propagated in the region of interest;

Step 110: calculating a frequency dispersion distribution diagram according to the ultrasonic echo signals;

Step 120: calculating a viscosity parameter according to the viscosity parameter of the ultrasonic echo signals, such as according to the frequency dispersion distribution diagram calculated by the ultrasonic echo signals; and

Step 130: performing quality control on the viscosity parameter according to the frequency dispersion distribution diagram.

For example, the viscosity parameter may be performed with quality control by directly displaying the frequency dispersion distribution diagram, by the frequency dispersion characteristic graph, by the viscosity quality control characteristic, or by the viscosity quality control score. It may be appreciated that the viscosity parameter may be performed with quality control by a combination of the above ways.

In some embodiments, in step 130, the viscosity quality control characteristic of the viscosity parameter may be obtained by the frequency dispersion distribution diagram, and the viscosity parameter may be quality controlled by the viscosity quality control characteristic. In some embodiments, the viscosity quality control characteristic may include any one or more of the characteristic quantities:
the effective frequency range for calculating the viscosity parameter;
the degree of matching when performing model fitting for the viscosity parameter according to the frequency dispersion distribution diagram;
the continuity of the frequency dispersion curve in the frequency dispersion distribution diagram;
the signal-to-noise ratio of the frequency dispersion distribution diagram; and
the different shear waves in multiple patterns in the frequency dispersion distribution diagram.

These viscosity quality control characteristics or characteristic quantities have been described in detail elsewhere herein and will not be repeated here.

In some examples, in step 130, the viscosity parameter may be performed with quality control by showing the viscosity quality control characteristic qualitatively or quantitatively, which is explained below.

In some embodiments, in step 130, a viscosity quality control characteristic may be drawn on the frequency dispersion distribution diagram to generate a frequency dispersion characteristic graph; and in step 130 the viscosity parameter may be quality controlled by displaying the frequency dispersion characteristic graph. The frequency dispersion characteristic graph may be generated by extracting, marking or enhancing the viscosity quality control characteristic in the frequency dispersion distribution diagram, or weakening the display background. For example, when the viscosity quality control characteristic includes the effective frequency range, the effective frequency range may be marked on the frequency dispersion distribution diagram, or the effective frequency range and the target frequency range for calculating the viscosity parameter may be marked on the frequency dispersion distribution diagram. For another example, when the viscosity quality control characteristic includes the degree of matching, the fitted line of the viscosity parameter may be obtained and plotted on the frequency dispersion distribution diagram, e.g. the fitted line is plotted on the frequency dispersion distribution diagram in dashed lines. For yet another example, when the viscosity quality control characteristic includes the continuity of the frequency dispersion curve in the frequency dispersion distribution diagram, only consecutive points in the frequency dispersion curve are connected on the frequency dispersion distribution diagram to draw the frequency dispersion curve, so that users can intuitively judge the continuity of the frequency dispersion curve. For still yet another example, when the viscosity quality control characteristic includes different shear waves in multiple patterns in the frequency dispersion distribution diagram, the frequency dispersion curve for each shear wave is extracted and plotted on the frequency dispersion distribution diagram so that users can see how many waves there has.

In some embodiments, in step 130, before drawing the viscosity quality control characteristic on the frequency dispersion distribution diagram, foreground feature enhancement or background fading may be performed on the frequency dispersion distribution diagram. For example, the background of the frequency dispersion distribution diagram is pre-configured, such as weakening the background image, strengthening the frequency dispersion curve of shear waves under each transmission mode, extracting the frequency dispersion curve of each shear wave, and displaying the frequency dispersion characteristic graph in the form of coordinate axis, etc.

In some embodiments, in step 130, the viscosity quality control information about the viscosity parameter may be displayed on the display 50 by the value characterizing the viscosity quality control characteristic, or the viscosity parameter may be performed with quality control by controlling the display 50 to display the value characterizing the viscosity quality control characteristic. The value of the viscosity quality control characteristic is a value obtained after quantization of the viscosity quality control characteristic, such as the number of wave modes, the ratio of main mode to total energy, an effective frequency range, accuracy of position calculation, a signal-to-noise ratio, a fitting correlation coefficient, and so on. The value obtained after quantization of the viscosity quality control characteristic may be a continuous value quantized, or may be classified, for example, the ratio of the main mode to the total energy may be 0-100%, or may be classified into low, medium and high.

For example, when the viscosity quality control characteristic includes the effective frequency range, the value of the effective frequency range may be displayed, or the value of the effective frequency range and the value of the target frequency range used for calculating the viscosity parameter may be displayed, or the overlapping degree of the effective frequency range and the target frequency range may be calculated and displayed. For another example, when the viscosity quality control characteristic includes the degree of matching, the fitted line of the viscosity parameter and the degree of fit between data used for fitting may be calculated and the degree of fit may be displayed. The degree of fit may include an average absolute difference value, a mean square error, a root mean square error, a coefficient of determination R2 or a correlation coefficient. For yet another example, when the viscosity quality control characteristic includes the continuity of the frequency dispersion curve in the frequency dispersion distribution diagram, the proportion of continuous or discontinuous segments in the frequency dispersion curve may be calculated and displayed. For still another example, when the viscosity quality control characteristic includes the signal-to-noise ratio of the frequency dispersion distribution diagram, the signal-to-noise ratio of the frequency dispersion distribution diagram may be calculated and displayed. For yet still another example, when the viscosity quality control characteristic includes the different shear waves in multiple patterns in the frequency dispersion distribution diagram, the number of different shear waves in multiple patterns in the frequency dispersion distribution diagram may be calculated and displayed, or the main shear wave may be determined and a degree of influence of other pattern waves on the main shear wave may be calculated and displayed. The degree of influence of other pattern waves on a main shear wave comprises a proportion of energy of other pattern waves or main shear wave.

In an embodiments, in step 130, a viscosity quality control score may be calculated according to the viscosity quality control characteristic, and the viscosity quality control information about the viscosity parameter may be displayed by the viscosity quality control score. In some embodiments, in step 130, the viscosity quality control score may be calculated according to the viscosity quality control characteristic as follows: performing weighted summation on each value characterizing the characteristic quantities to obtain the viscosity quality control score. In some embodiments, when the viscosity quality control characteristic includes the effective frequency range, the value characterizing the characteristic quantities may be the overlapping degree of the effective frequency range and the target frequency range; when the viscosity quality control characteristic includes the degree of matching, the value characterizing the characteristic quantities may be the fitted line of the viscosity parameter and the degree of fit between data used for fitting; when the viscosity quality control characteristic includes the continuity of the frequency dispersion curve in the frequency dispersion distribution diagram, the value characterizing the characteristic quantities may be the proportion of continuous or discontinuous segments in the frequency dispersion curve; when the viscosity quality control characteristic includes the signal-to-noise ratio of the frequency dispersion distribution diagram, the value characterizing the characteristic quantities may be the signal-to-noise ratio of the frequency dispersion distribution diagram; and when the viscosity quality control characteristic includes the different shear waves in multiple patterns in the frequency dispersion distribution diagram, the value characterizing the characteristic quantities may be the degree of influence of other pattern waves on the main shear wave. The degree of influence of other pattern waves on the main shear wave may include the proportion of energy of other pattern waves or the main shear wave.

In some embodiments, in step 130, the viscosity quality control information about the viscosity parameter may be displayed by the viscosity quality control score as follows: in step 130, the viscosity quality control distribution diagram of the region of interest may be generated according to the viscosity quality control score of each point in the region of interest; and the viscosity quality control distribution diagram may be controlled to display.

In some embodiments, in step 130, a region of the viscosity parameter distribution diagram where the viscosity quality control information fails to meet a predetermined requirement may be emptied, the viscosity parameter distribution diagram may be generated based on the viscosity parameter of each point in the region of interest. For example, each value characterizing the characteristic quantities may be weighted and summed to obtain the viscosity quality control score, and the viscosity quality control distribution diagram of the region of interest may be generated according to the viscosity quality control score of each point in the region of interest.

In some embodiments, in step 130, the viscosity parameter may be performed with quality control by controlling to display the frequency dispersion distribution diagram. In some instances, the frequency dispersion distribution diagram may represent the relationship between shear wave propagation properties and frequency, such as the relationship between shear wave propagation velocity and frequency, with the horizontal coordinate being frequency and the vertical coordinate being velocity, it being understood that the horizontal and vertical coordinates may be substituted for each other. Alternatively, the shear wave velocity and frequency may be calculated to obtain the slowness (inverse of velocity) versus frequency or wave number (ratio of velocity to frequency) versus frequency as the frequency dispersion distribution diagram.

In some embodiments, in step 130, one or more of the frequency dispersion distribution diagram, the frequency dispersion characteristic graph, the viscosity quality control characteristic, the viscosity quality control score, and the viscosity quality control distribution diagram may be displayed in combination with any one or more of a conventional B-mode image, an elasticity image, an elasticity quality control distribution diagram, or the viscosity parameter distribution diagram (which may also be referred to as a viscosity image) for a user to comprehensively judge the quality of the imaging. The elasticity image may be generated according to elasticity parameters of each point in the region of interest. The elasticity parameters may also include various types according to different elasticity imaging methods; for example, the elasticity parameters may be a strain, a strain ratio and a strain rate for conventional pressed type elasticity imaging, or be a shear wave velocity, a shear wave velocity ratio, a Young's modulus, a shear modulus, a Young's modulus ratio, a shear modulus ratio and a shear wave propagation distance for shear wave elasticity imaging. In this embodiment, the parameters may include, but be not limited to: strain-to-strain ratio, strain-time curve, shear wave velocity-to-shear wave velocity ratio, elastic modulus-to-elastic modulus ratio, elastic histogram statistics, and so on. An elasticity quality control distribution diagram is a graph used to quality control an elasticity image.

The present disclosure is illustrated with reference to various exemplary embodiments. However, those skilled in the art may recognize that the exemplary embodiments can be changed and modified without departing from the scope of the present disclosure. For example, various operation steps and components used to execute the operation steps may be implemented in different ways (for example, one or more steps may be deleted, modified, or combined into other steps) according to specific application(s) or any number of cost functions associated with the operation of the system.

In addition, as understood by those skilled in the art, the principles herein may be reflected in a computer program product on a computer-readable storage medium that is preloaded with computer-readable program code. Any tangible, non-temporary computer-readable storage medium can be used, including magnetic storage devices (hard disks, floppy disks, etc.), optical storage devices (CD-ROMs, DVDs, Blu Ray disks, etc.), flash memory and/or the like. The computer program instructions may be loaded onto a general purpose computer, a special purpose computer, or other programmable data processing device to form a machine, so that these instructions executed on a computer or other programmable data processing device can form a device that realizes a specified function. These computer program instructions may also be stored in a computer-readable memory that can instruct a computer or other programmable data processing device to run in a specific way, so that the instructions stored in the computer-readable memory can form a manufacturing product, including a realization device to achieve a specified function. The computer program instructions may also be loaded onto a computer or other programmable data processing device to execute a series of operating steps on the computer or other programmable device to produce a computer-implemented process, so that instructions executed on the computer or other programmable device can provide steps for implementing a specified function.

Although the principles herein have been shown in various embodiments, many modifications to structures, arrangements, proportions, elements, materials, and components that are specifically adapted to specific environmental and operational requirements may be used without deviating from the principles and scope of the present disclosure. These and other modifications and amendments will be included in the scope of the present disclosure.

The foregoing specific description has been illustrated with reference to various embodiments. However, those skilled in the art will recognize that various modifications and changes can be made without departing from the scope of the present disclosure. Accordingly, the present disclosure is illustrative rather than restrictive, and all such modifications will be included in its scope. Similarly, there are solutions to these and other advantages and problems of the various embodiments as described above. However, the benefits, the advantages, solutions to problems, and any elements that can produce them or make them more explicit should not be interpreted as critical, required, or necessary one. The term “comprise” and any other variations thereof used herein are non-exclusive; accordingly, a process, method, article or device that includes a list of elements may include not only these elements, but also other elements that are not explicitly listed or are not part of said process, method, article or device. In addition, the term “coupling” and any other variations thereof as used herein may refer to physical, electrical, magnetic, optical, communication, functional, and/or any other connection.

Those skilled in the art will realize that many changes can be made to the details of the above embodiments without departing from the basic principles of the present disclosure. The scope of the present disclosure shall therefore be determined in accordance with the following claims.

What is claimed is:

1. A viscosity quality control method, comprising:
transmitting ultrasonic waves for detecting shear waves to obtain ultrasonic echo signals, the shear waves being propagated in a region of interest;
calculating a frequency dispersion distribution diagram according to the ultrasonic echo signals;
calculating a viscosity parameter according to the frequency dispersion distribution diagram;
obtaining a viscosity quality control characteristic of the viscosity parameter according to the frequency dispersion distribution diagram; and
displaying viscosity quality control information about the viscosity parameter according to the viscosity quality control characteristic.

2. The viscosity quality control method according to claim 1, wherein the viscosity quality control characteristic comprises any one or more of the following characteristic quantities:
an effective frequency range for calculating the viscosity parameter;
a degree of matching when performing model fitting for the viscosity parameter according to the frequency dispersion distribution diagram;
a continuity of a frequency dispersion curve in the frequency dispersion distribution diagram;
a signal-to-noise ratio of the frequency dispersion distribution diagram; and
different shear waves in multiple patterns in the frequency dispersion distribution diagram.

3. The viscosity quality control method according to claim 2, wherein displaying viscosity quality control information about the viscosity parameter according to the viscosity quality control characteristic comprises:
plotting the viscosity quality control characteristic on the frequency dispersion distribution diagram to generate a frequency dispersion characteristic graph; and
displaying the frequency dispersion characteristic graph.

4. The viscosity quality control method according to claim 3, wherein plotting the viscosity quality control characteristic on the frequency dispersion distribution diagram to generate a frequency dispersion characteristic graph comprises:
marking the effective frequency range on the frequency dispersion distribution diagram when the viscosity quality control characteristic comprises the effective frequency range, or marking the effective frequency range and a target frequency range used for calculating the viscosity parameter on the frequency dispersion distribution diagram;
and/or
obtaining a fitted line in calculation of the viscosity parameter when the viscosity quality control characteristic comprises the degree of matching, and plotting the fitted line on the frequency dispersion distribution diagram;
and/or
connecting only continuous points of the frequency dispersion curve in the frequency dispersion distribution diagram so as to plot the frequency dispersion curve when the viscosity quality control characteristic comprises the continuity of the frequency dispersion curve in the frequency dispersion distribution diagram;
and/or
extracting frequency dispersion curves for respective shear waves in multiple patterns and plotting the extracted dispersion curves on the frequency dispersion distribution diagram when the viscosity quality control characteristic comprises different shear waves in multiple patterns in the frequency dispersion distribution diagram.

5. The viscosity quality control method according to claim 3, further comprising: performing foreground feature enhancement or background fading process on the frequency dispersion distribution diagram before plotting the viscosity quality control characteristic on the frequency dispersion distribution diagram.

6. The viscosity quality control method according to claim 2, wherein displaying the viscosity quality control information about the viscosity parameter according to the viscosity quality control characteristic comprises:
displaying the viscosity quality control information about the viscosity parameter by a value characterizing the viscosity quality control characteristic.

7. The viscosity quality control method according to claim 6, wherein displaying the viscosity quality control information about the viscosity parameter by a value characterizing the viscosity quality control characteristic comprises:
when the viscosity quality control characteristic comprises the effective frequency range, displaying a value of the effective frequency range, or displaying a value of the effective frequency range and a value of a target frequency range used for calculating the viscosity parameter, or calculating and displaying an overlapping degree of the effective frequency range and a target frequency range;
and/or
calculating a fitted line of the viscosity parameter and a degree of fit between data used for fitting when the viscosity quality control characteristic comprises the degree of matching, and displaying the degree of fit comprising an average absolute difference value, a mean square error, a root mean square error, a coefficient of determination R 2 or a correlation coefficient;
and/or
calculating and displaying a proportion of continuous or discontinuous segments in the frequency dispersion curve when the viscosity quality control characteristic comprises the continuity of the frequency dispersion curve in the frequency dispersion distribution diagram;
and/or
calculating and displaying a signal-to-noise ratio of the frequency dispersion distribution diagram when the viscosity quality control characteristic comprises the signal-to-noise ratio of the frequency dispersion distribution diagram;
and/or
when the viscosity quality control characteristic comprises the different shear waves in multiple patterns in the frequency dispersion distribution diagram, calculating and displaying a number of the different shear waves in multiple patterns in the frequency dispersion distribution diagram, or, determining a main shear wave and calculating and displaying a degree of influence of other pattern waves on the main shear wave, wherein the degree of influence of other pattern waves on the main shear wave comprises a proportion of energy of other pattern waves or main shear wave.

8. The viscosity quality control method according to claim 2, wherein displaying the viscosity quality control information about the viscosity parameter according to the viscosity quality control characteristic comprises:
calculating a viscosity quality control score according to the viscosity quality control characteristic; and
displaying the viscosity quality control information about the viscosity parameter by the viscosity quality control score.

9. The viscosity quality control method according to claim 8, wherein calculating a viscosity quality control score according to the viscosity quality control characteristic comprises:
performing weighted summation on each value characterizing the characteristic quantities to obtain the viscosity quality control score;
wherein, the value characterizing the characteristic quantities is an overlapping degree of the effective frequency range and a target frequency range when the viscosity quality control characteristic comprises the effective frequency range, or the value characterizing the characteristic quantities is a fitted line of the viscosity parameter and a degree of fit between data used for fitting when the viscosity quality control characteristic comprises the degree of matching, or the value characterizing the characteristic quantities is a proportion of continuous or discontinuous segments in the frequency dispersion curve when the viscosity quality control characteristic comprises a continuity of the frequency dispersion curve in the frequency dispersion distribution diagram, or the value characterizing the characteristic quantities is a signal-to-noise ratio of the frequency dispersion distribution diagram when the viscosity quality control characteristic comprises the signal-to-noise ratio of the frequency dispersion distribution diagram; or the value characterizing the characteristic quantities is a degree of influence of other pattern waves on a main shear wave when the viscosity quality control characteristic comprises different shear waves in multiple patterns in the frequency dispersion distribution diagram, the degree of influence of other pattern waves on a main shear wave comprises a proportion of energy of other pattern waves or main shear wave.

10. The viscosity quality control method according to claim 8, wherein displaying the viscosity quality control information about the viscosity parameter by the viscosity quality control score comprises:
generating a viscosity quality control distribution diagram of the region of interest according to the viscosity quality control score of each point in the region of interest; and
displaying the viscosity quality control distribution diagram.

11. The viscosity quality control method according to claim 1, wherein displaying the viscosity quality control information about the viscosity parameter according to the viscosity quality control characteristic comprises emptying a region in a viscosity parameter distribution diagram where the viscosity quality control information fails to meet a predetermined requirement according to the viscosity quality control characteristic; and the viscosity parameter distribution diagram is generated based on the viscosity parameter of each point in the region of interest.

12. The viscosity quality control method, comprising:
transmitting ultrasonic waves for detecting shear waves to a region of interest to obtain ultrasonic echo signals, the shear waves being propagating in the region of interest;
calculating a frequency dispersion distribution diagram according to the ultrasonic echo signals;
calculating a viscosity parameter according to the frequency dispersion distribution diagram; and
performing quality control on the viscosity parameter according to the frequency dispersion distribution diagram.

13. The viscosity quality control method according to claim 12, wherein performing quality control on the viscosity parameter according to the frequency dispersion distribution diagram comprises: performing quality control on the viscosity parameter by displaying the frequency dispersion distribution diagram.

14. The viscosity quality control method according to claim 12, wherein performing quality control on the viscosity parameter according to the frequency dispersion distribution diagram comprises:
obtaining a viscosity quality control characteristic of the viscosity parameter according to the frequency dispersion distribution diagram;
plotting the viscosity quality control characteristic on the frequency dispersion distribution diagram to generate a frequency dispersion characteristic graph; and
performing quality control on the viscosity parameter by displaying the frequency dispersion characteristic graph.

15. The viscosity quality control method according to claim 12, wherein performing quality control on the viscosity parameter according to the frequency dispersion distribution diagram comprises:
obtaining a viscosity quality control characteristic of the viscosity parameter according to the frequency dispersion distribution diagram; and
performing quality control on the viscosity parameter by displaying a value characterizing the viscosity quality control characteristic.

16. The viscosity quality control method according to claim 12, wherein performing quality control on the viscosity parameter according to the frequency dispersion distribution diagram comprises:
obtaining a viscosity quality control characteristic of the viscosity parameter according to the frequency dispersion distribution diagram;
calculating a viscosity quality control score according to the viscosity quality control characteristic; and
displaying viscosity quality control information about the viscosity parameter by the viscosity quality control score.

17. The viscosity quality control method according to claim 12, wherein performing quality control on the viscosity parameter according to the frequency dispersion distribution diagram comprises:
obtaining a viscosity quality control characteristic of the viscosity parameter according to the frequency dispersion distribution diagram; and
emptying a region in a viscosity parameter distribution diagram where the viscosity quality control information fails to meet a predetermined requirement according to the viscosity quality control characteristic, wherein the viscosity parameter distribution diagram is generated based on the viscosity parameter of each point in the region of interest.

18. The viscosity quality control method according to claim 14, wherein the viscosity quality control characteristic comprises any one or more of the following characteristic quantities:
an effective frequency range for calculating the viscosity parameter;
a degree of matching when performing model fitting for the viscosity parameter according to the frequency dispersion distribution diagram;
a continuity of a frequency dispersion curve in the frequency dispersion distribution diagram;
a signal-to-noise ratio of the frequency dispersion distribution diagram; and
different shear waves in multiple patterns in the frequency dispersion distribution diagram.
